(19) United States
(12) Patent Application Publication
(10) Pub. No.: US 2012/0029611 A1
(43) Pub. Date: Feb. 2, 2012

(54) Stent Graft System and Method of Use

(75) Inventors: JENNA WEIDMAN, Santa Rosa, CA (US); SHANNON MITCHELL, Santa Rosa, CA (US); ANDREW KIEHL, Santa Rosa, CA (US)

(73) Assignee: Medtronic Vascular, Inc., Santa Rosa, CA (US)

(21) Appl. No.: 12/845,117

(22) Filed: Jul. 28, 2010

(51) Int. Cl. A61F 2/06

(52) U.S. Cl. 623/1.13

(57) ABSTRACT

A stent graft system and method of use including a stent graft system with a first stent graft component and a second stent graft component. A first tubular graft of the first stent graft component has an integral first body and first leg, with the first body defining a first crown opening and a first substantially elliptical opening. The second body of the second stent graft component is disposed in the first body of the first stent graft component with the second leg of the second stent graft component disposed through the first substantially elliptical opening of the first stent graft component with the perimeter of the first elliptical opening of the first stent graft component in contact with the second body of the second stent graft component.

### TECHNICAL FIELD

The technical field of this disclosure is medical implantation devices, particularly, a stent graft system and method of use.

### BACKGROUND

Stent grafts have been developed for the treatment of abdominal aortic aneurysms. An abdominal aortic aneurysm is a bulge that forms in the wall of the abdominal aorta, which is the main vessel of the arterial system of the body that extends through the abdomen. Abdominal aortic aneurysms can have various causes, such as high blood pressure, accident, or atherosclerosis, and can lose elasticity over time and rupture under normal blood pressure. A stent graft is typically a woven tube (graft) supported by one or more tubular metal or plastic stents. The stent graft is placed inside and spanning an aneurysm to exclude the abdominal aortic aneurysm from normal blood flow and reduces pressure on the aneurysmal vessel.

Presently, stent grafts include a number of pieces that are assembled within the patient to provide the finished stent graft assembly. The main larger piece includes a trunk with two descending limbs: an iliac limb and a contralateral limb. The contralateral limb can be shorter than or equal in length to the iliac limb, but both have a length extending from the trunk. A limb segment is deployed in the contralateral limb to extend between the contralateral limb and the iliac artery. Iliac extension stent grafts can be used to extend the iliac limb and/or the limb segment as required. Unfortunately, the number of parts required for the stent graft increases manufacturing and inventory costs. The stent graft pieces must be manufactured and stocked in a number of lengths and diameters at the supplier and at the hospital, so each separate part greatly increases the complexity of the therapy delivery.

The present stent grafts also present challenges during deployment. The typical contralateral limb has a small opening, making it difficult to locate and feed in a guidewire for guiding deployment of the limb segment in the contralateral limb. In addition, the thickness of the graft material when compressed creates bulk (volume) which limits the amount of graft material and thereby the diameter of the stent graft delivery configuration that can be compressed into a given size catheter, limiting flexibility and deployment location access.

It would be desirable to have a stent graft system and method of use that would overcome the above disadvantages.

### SUMMARY

One aspect according to the present invention provides a stent graft system including a first stent graft component and a second stent graft component. The first stent graft component includes a first tubular graft having an integral first body and first leg, the first body having a first longitudinal axis, the first body defining a first crown opening and a first substantially elliptical opening, the first substantially elliptical opening being oriented at a first acute angle to the first longitudinal axis. The second stent graft component includes a second tubular graft having an integral second body and second leg, the second body having a second longitudinal axis, the second body defining a second crown opening and a second substantially elliptical opening, the second substantially elliptical opening being oriented at a second acute angle to the second longitudinal axis. The second body is disposed in the first body, and the second leg is disposed through the first substantially elliptical opening with the perimeter of the first elliptical opening in contact with the second body.

Another aspect according to the present invention provides a stent graft including a tubular graft having an integral body and leg, the body having a longitudinal axis, the body defining a crown opening and a substantially elliptical opening, the substantially elliptical opening being oriented at an acute angle to the longitudinal axis; an elliptical opening stent operably connected to the substantially elliptical opening; and a crown stent operably connected around the crown opening.

Another aspect according to the present invention provides a method of use of a stent graft system including positioning a first stent graft component in a first compressed configuration at a deployment site. The first stent graft component includes a first tubular graft having an integral first body and first leg, the first body having a first longitudinal axis, the first body defining a first crown opening and a first substantially elliptical opening, the first substantially elliptical opening being oriented at a first acute angle to the first longitudinal axis; and a first elliptical opening stent operably connected to the first substantially elliptical opening. The method further includes releasing the first stent graft component from the first compressed configuration to expand at the deployment site; and advancing a second stent graft component in a second compressed configuration through the first substantially elliptical opening to the deployment site. The second stent graft component includes a second tubular graft having an integral second body and second leg, the second body having a second longitudinal axis, the second body defining a second crown opening and a second substantially elliptical opening, the second substantially elliptical opening being oriented at a second acute angle to the second longitudinal axis; and a second elliptical opening stent operably connected to the substantially elliptical opening. The method further includes releasing the second stent graft component from the second compressed configuration to expand with the second body within the first body and the second leg disposed through the first substantially elliptical opening with the perimeter of the first substantially elliptical opening in contact with the second body.

The foregoing and other features and advantages will become further apparent from the following detailed description of the presently preferred embodiments, read in conjunction with the accompanying drawings

### BRIEF DESCRIPTION OF THE DRAWINGS

FIG. 1 is a side view of a stent graft component;

FIG. 2 is a side view of a stent graft system;

FIG. 3 is a side view of an assembled stent graft system;

FIG. 4 is a flow chart of a method of creating a stent graft system;

FIGS. 5A-5F are cut-away side views of a method of creating a stent graft system;

FIG. 6 is a close-up view of an elliptical opening stent for a stent graft system;

FIGS. 7-9 are additional embodiments of a stent graft component; and

FIGS. 10A-10D are various views of an exemplary stent graft system;

FIGS. 11A & 11B are side views of another exemplary stent graft system; and

FIG. 12 is a side view of another exemplary assembled stent graft system.

### DETAILED DESCRIPTION

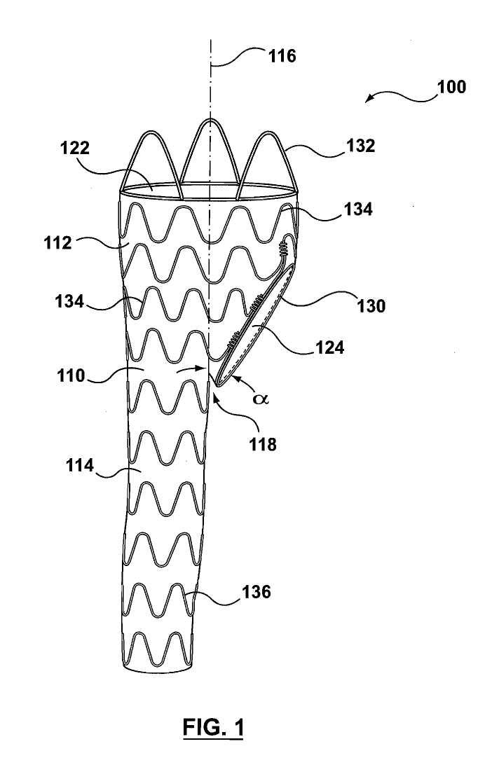

FIG. 1 is a side view of a stent graft component. The first stent graft component 100 includes a tubular graft 110, an elliptical opening stent 130, a crown stent 132, body stents 134, and leg stents 136. The tubular graft 110 has an integral body 112 and leg 114. In this example, the body 112 has a longitudinal axis 116 and a leg intersection point 118 near the longitudinal axis 116 where the leg 114 meets the body 112. The body 112 defines a crown opening 122 and a substantially elliptical opening 124. The substantially elliptical opening 124 is oriented at an acute angle α to the longitudinal axis 116. The elliptical opening stent 130 is operably connected to the substantially elliptical opening 124, the crown stent 132 is operably connected around the crown opening 122, the body stents 134 are operably connected around the body 112, and the leg stents 136 are operably connected around the leg 114. In this example, the elliptical opening stent 130 is attached to several of the body stents 134. In one embodiment, the acute angle α is between 20 degrees and 70 degrees. In another embodiment, the acute angle α is about 30 degrees. The longitudinal axis 116 is generally parallel to the sides of the body 112 and need not be located in the center of the body 112. The body 112 can be symmetrical in cross section, or can have the larger portion aligned with the leg 114 or the substantially elliptical opening 124.

Those skilled in the art will appreciate that the stent graft component can be adapted as desired for a particular application. Substantially elliptical as used herein in conjunction with the substantially elliptical opening is defined as having a generally elliptical shape, such as a regular or irregular ellipse, circle, oval, egg shape, eggplant shape, tear drop shape, or the like. The perimeter of the generally elliptical shape can include minor deviations from the generally elliptical shape as desired for a particular application, e.g., the perimeter can be peanut shaped. In one embodiment, the elliptical opening stent extends around the whole perimeter of the substantially elliptical opening. In another embodiment, the elliptical opening stent extends partially around the perimeter of the substantially elliptical opening. In yet another embodiment, the elliptical opening stent includes a number of separate stent segments extending partially or fully around the perimeter of the substantially elliptical opening. In yet another embodiment, the elliptical opening stent is one of the other stents, such as the body stent, with a portion of the body stent urging open the substantially elliptical opening without necessarily following the perimeter of the substantially elliptical opening. The elliptical opening stent as used herein is defined as any stent or portion of a stent that urges open the substantially elliptical opening when the stent graft component is in an expanded configuration. In yet another embodiment, no elliptical opening stent is provided around the substantially elliptical opening.

The stent graft component can be made of any suitable biocompatible materials. Exemplary materials for the elliptical opening stent 130, crown stent 132, body stent 134, and leg stent 136 include stainless steels, such as 316L stainless steel; shape memory alloys, such as nitinol; and nickel-cobalt-chromium-molybdenum alloy, such as MP35N® alloy available from SPS Technologies, Inc., of Jenkintown, Pa.; titanium; tantalum; or the like. Those skilled in the art will appreciate that various stent configurations are possible. In one alternative, the stent is woven into the graft material. In another alternative, the stent is added when the stent graft component is at the deployment site, by injecting a hardenable material into recesses within the stent graft component.

Exemplary materials for the tubular graft 110 include biocompatible, blood-impermeable graft membranes, such as polyester, polyethylene, expanded polytetrafluoroethylene (ePTFE), polyurethane, propylene, nylon, and the like. In one embodiment, the material for the tubular graft 110 can be thinner than conventional graft material because the two bodies of the stent graft components overlap in the assembled stent graft system.

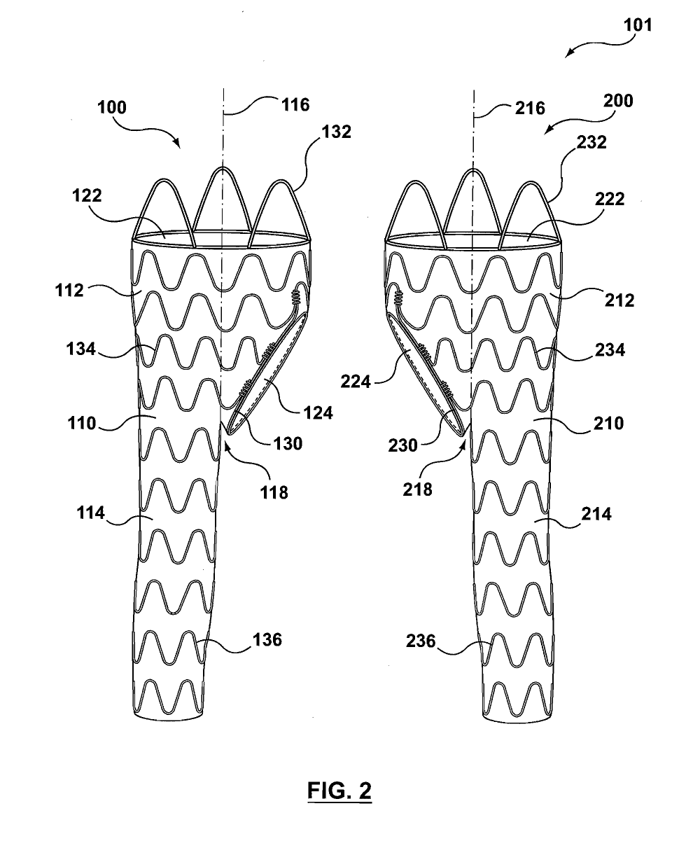

FIG. 2, in which like elements share like reference numbers with FIG. 1, is a side view of a stent graft system. The stent graft system 101 includes first stent graft component 100 and second stent graft component 200. In this example, the first stent graft component 100 is identical to the second stent graft component 200, although in other embodiments the stent graft components can be different in size and/or shape.

The second stent graft component 200 includes a tubular graft 210, an elliptical opening stent 230, a crown stent 232, body stents 234, and leg stents 236. The tubular graft 210 has an integral body 212 and leg 214. The body 212 has a longitudinal axis 216 and a leg intersection point 218 on the longitudinal axis 216 where the leg 214 meets the body 212. The body 212 defines a crown opening 222 and a substantially elliptical opening 224. The elliptical opening stent 230 is operably connected to the substantially elliptical opening 224, the crown stent 232 is operably connected around the crown opening 222, the body stents 234 are operably connected around the body 212, and the leg stents 236 are operably connected around the leg 214. In this example, the elliptical opening stent 230 is attached to several of the body stents 234. Also in this example, the crown stent 132 and the crown stent 232 each includes an odd number of peaks beyond the graft material of the body 112 and body 212, respectively, so that when the components are combined the peaks are complementary and follow the whole perimeter of the crown opening to form a continuous row of peaks when the stent graft system is assembled. Those skilled in the art will appreciate that the number and configuration of the crown stent peaks can be selected as desired for a particular application. In another embodiment, the number of peaks can be even. In one embodiment, the peaks are aligned so the peaks of one stent graft component overlap the peaks of the other stent graft component, providing an overlapping double row of peaks when the stent graft system is assembled.

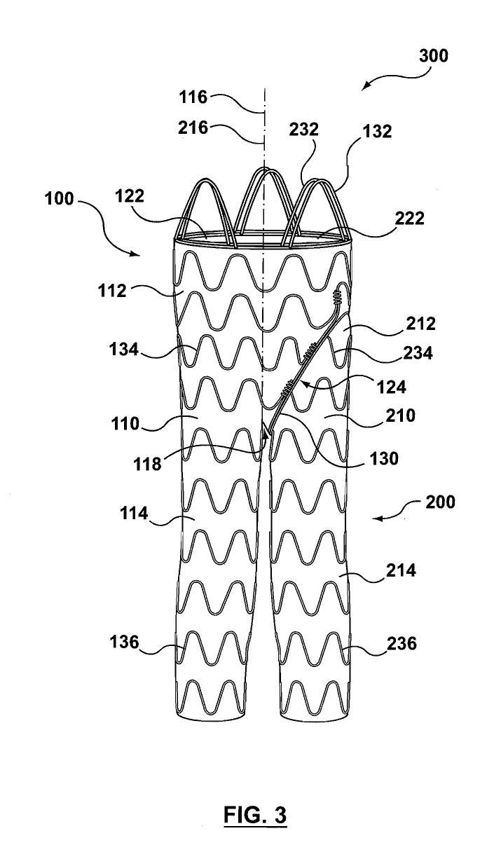

FIG. 3, in which like elements share like reference numbers with FIG. 2, is a side view of an assembled stent graft system. The assembled stent graft system 300 has body 212 of the second stent graft component 200 disposed in the body 112 of the first stent graft component 100. In this example, the crown opening 222 of the second stent graft component 200 substantially aligned with the crown opening 122 of the first stent graft component 100. In one embodiment, the body 212 is held in place relative to the body 112 by friction of the interference fit between the body 212 and the body 112. In one embodiment, the body 212 is held in place relative to the body 112 by operably connecting the body 212 to the body 112 with fasteners, such as sutures, staples, hooks, anchors, adhesives, rivets, screws, or the like. The second leg 214 of the second stent graft component 200 is disposed through the substantially elliptical opening 124 of the first stent graft component 100 with the perimeter of the first elliptical opening 124 in contact with the body 212 of the second stent graft component 200. The longitudinal axis 116 of the first stent graft component 100 is substantially aligned with the longitudinal axis 216 of the second stent graft component 200. In this example, the peaks of the crown stent 132 and the crown stent 232 alternate around the perimeter of the crown opening. Substantially aligned as used herein in conjunction with the crown opening 222 and crown opening 122 is defined as being generally coplanar, with allowance for minor angular and/or axial (dimensional) offsets that do not affect function. In another embodiment, the body of one of the stent graft components is longer than the body of the other stent graft component, so that the crown openings are not aligned. This allows the crown stent of one stent graft component to contact the aorta at a position longitudinally offset from the position where the crown stent of the other stent graft component contacts the aorta. In yet another embodiment, the crown openings are not aligned due to the particular placement of the individual stent graft components even though the body of the two stent graft components are the same length.

Those skilled in the art will appreciate that additional stent graft components can be added to the stent graft system as desired for a particular application. In one example, a seal graft, such as a tubular graft with or without supporting stents, can be placed within the body of the second stent graft component and the leg of the first stent graft component to seal the joint where the second stent graft component meets the first stent graft component. The seal graft extends across the substantially elliptical opening of the second stent graft component to provide the seal for blood flow from the body of the second stent graft component into the leg of the first stent graft component and out to the vessel.

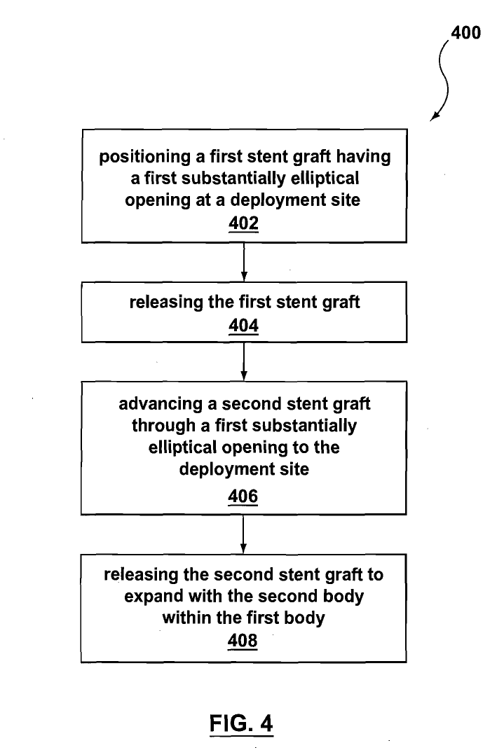

FIG. 4 is a flow chart of the steps of a method of creating a stent graft system. The method 400 includes positioning a first stent graft component in a first compressed configuration at a deployment site 402; releasing the first stent graft component from the first compressed configuration to expand at the deployment site 404; advancing a second stent graft component in a second compressed configuration through the first substantially elliptical opening to the deployment site 406; and releasing the second stent graft component from the second compressed configuration to expand with the second body within the first body 408 and the second leg disposed through the first substantially elliptical opening with the perimeter of the first elliptical opening in contact with the second body.

The first stent graft component includes a first tubular graft having an integral first body and first leg, the first body having a first longitudinal axis, the first body defining a first crown opening and a first substantially elliptical opening, the first substantially elliptical opening being oriented at a first acute angle to the first longitudinal axis; and a first elliptical opening stent operably connected to the first substantially elliptical opening.

The second stent graft component includes a second tubular graft having an integral second body and second leg, the second body having a second longitudinal axis, the second body defining a second crown opening and a second substantially elliptical opening, the second substantially elliptical opening being oriented at a second acute angle to the second longitudinal axis; and a second elliptical opening stent operably connected to the second substantially elliptical opening.

In one embodiment, the method 400 can optionally include attaching the first stent graft component to the second stent graft component with a fastener, such as sutures, staples, hooks, anchors, adhesives, rivets, screws, or the like. In one embodiment, positioning a first stent graft component 402 includes positioning the first stent graft component through the vasculature, such as positioning the first stent graft component in a first compressed configuration at a deployment site through a first common iliac artery, and advancing a second stent graft component 408 includes advancing the second stent graft component from a second common iliac artery through the first substantially elliptical opening.

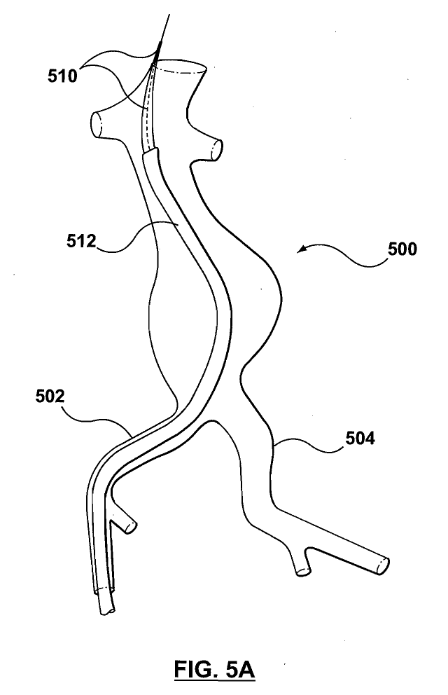

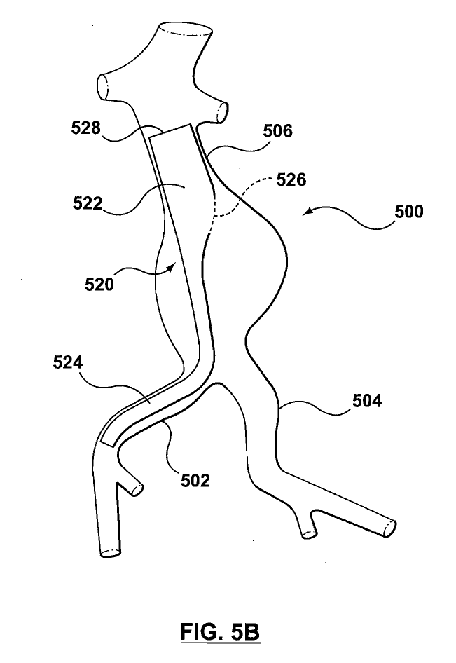

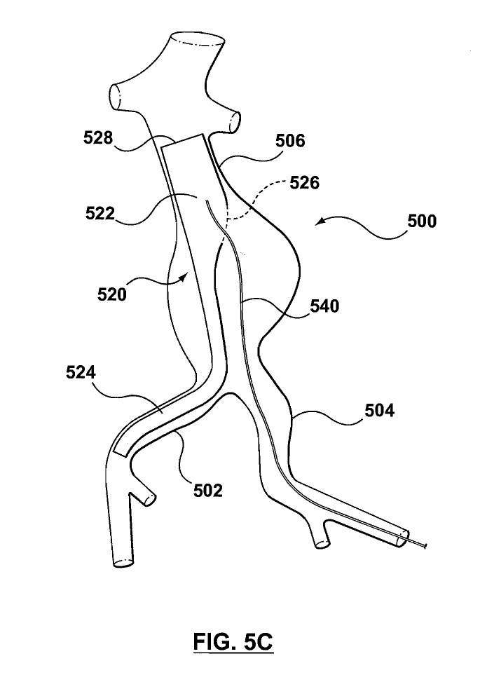

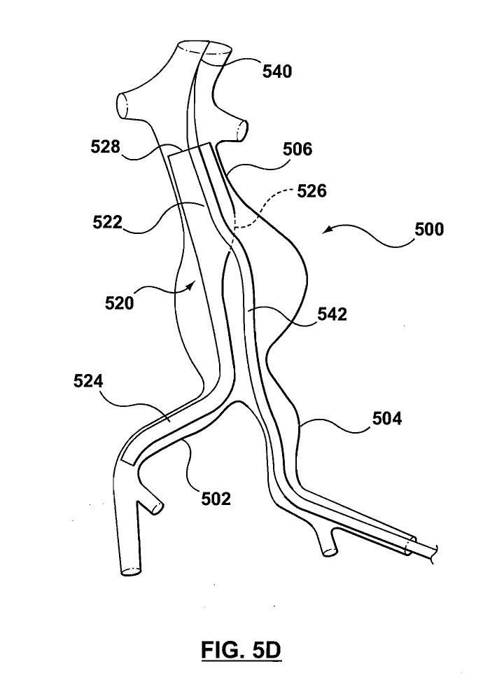

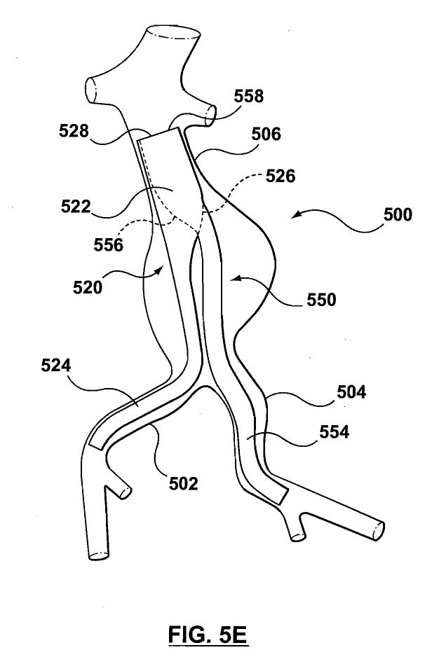

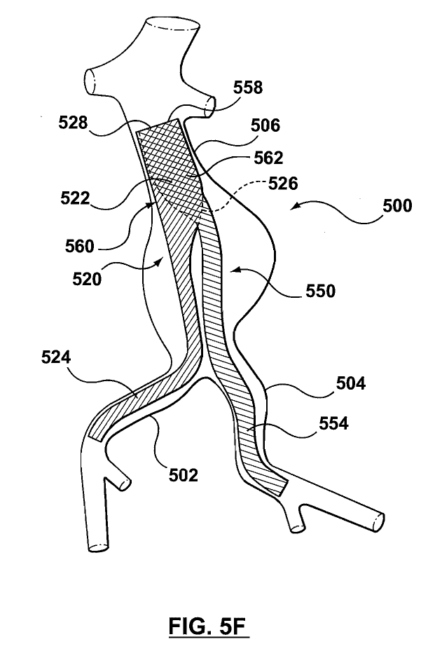

FIGS. 5A-5F, in which like elements share like reference numbers, are cut-away side views of the steps of a method of creating a stent graft system. In this example, the stent graft system is being deployed in an aortic aneurysm.

Referring to FIG. 5A, a guidewire 510 is advanced through the common iliac artery 502 and into the aortic aneurysm 500. A stent graft delivery system 512 holding a first stent graft component in a compressed configuration is advanced over the guidewire 510 to position the first stent graft component at the deployment site in the aortic aneurysm 500.

Referring to FIG. 5B, the stent graft delivery system releases the first stent graft component 520 from the compressed configuration to expand at the deployment site. In one embodiment, the stent graft delivery system releases the first stent graft component 520 by retracting a sheath holding the first stent graft component 520 in the compressed configuration. The guidewire can be retracted from the aortic aneurysm 500 after the first stent graft component 520 expands. The body 522 of the first stent graft component 520 is located at the neck 506 of the aortic aneurysm 500 and the leg 524 of the first stent graft component 520 is located in the common iliac artery 502. The first stent graft component 520 includes a substantially elliptical opening 526 and a crown opening 528.

Referring to FIG. 5C, a guidewire 540 is advanced through the common iliac artery 504, the substantially elliptical opening 526, and the crown opening 528.

Referring to FIG. 5D, a stent graft delivery system 542 holding a second stent graft component in a compressed configuration is advanced over the guidewire 540 to position the second stent graft component at the deployment site in the aortic aneurysm 500.

Referring to FIG. 5E, the stent graft delivery system releases the second stent graft component 550 from the compressed configuration to expand at the deployment site. In one embodiment, the stent graft delivery system releases the second stent graft component 550 by retracting a sheath holding the second stent graft component 550 in the compressed configuration. The guidewire can be retracted from the aortic aneurysm 500 after the second stent graft component 550 expands. The body (not shown) of the second stent graft component 550 is located at the neck 506 of the aortic aneurysm 500 and the leg 554 of the second stent graft component 550 is located in the common iliac artery 504. The second stent graft component 550 includes a substantially elliptical opening 556 (illustrated by dashed lines within the body 522 of the first stent graft component 520) and a crown opening 558.

The first stent graft component 520 and second stent graft component 550 fit together to provide a flow path past the aortic aneurysm 500 to the common iliac arteries 502, 504. The body (not shown) of the second stent graft component 550 is located within the body 522 of the first stent graft component 520. In this example, the crown opening 558 of the second stent graft component 550 is substantially aligned with and inside of the crown opening 528 of the first stent graft component 520. The leg 554 of the second stent graft component 550 is disposed through the first substantially elliptical opening 526 of the first stent graft component 520 with the perimeter of the first substantially elliptical opening in contact with the body (not shown) of the second stent graft component 550. The substantially elliptical opening 556 of the second stent graft component 550 is positioned to align with the lumen of the leg 524 of the first stent graft component 520. In one embodiment, the first stent graft component 520 can be fastened to the second stent graft component 550 as desired to preclude leakage, with fasteners, such as sutures, staples, hooks, anchors, adhesives, rivets, screws, or the like. In another embodiment, a stent graft sleeve can be installed within the first stent graft component 520 and/or the second stent graft component 550 to provide an added seal.

Referring to FIG. 5F, the deployed stent graft system 560 includes the first stent graft component 520 and the second stent graft component 550. In one embodiment, the material of the tubular graft can be thinner than the material of conventional stent grafts because the bodies of the stent graft components 520, 550 overlap at the neck 506 of the aortic aneurysm 500 as illustrated by the hashed portion 562. Those skilled in the art will appreciate that different deployment approaches are possible as desired for a particular application. For example, in another embodiment, the stent graft system can be deployed without the use of guidewires. In yet another embodiment, the stent graft system can be deployed through the common iliac artery or arteries.

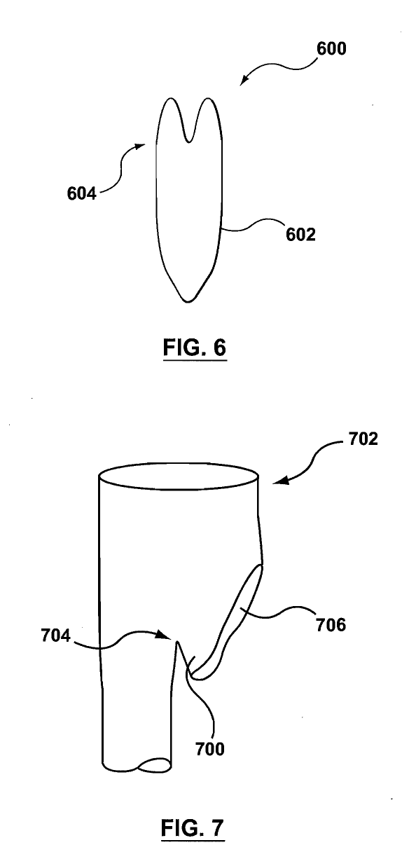

FIG. 6 is a close-up view of an elliptical opening stent for a stent graft system. The elliptical opening stent holds the substantially elliptical opening in the stent graft component open to receive the guidewire and stent graft delivery system during deployment and to seal with the other stent graft component after deployment. The elliptical opening stent has a small crossing profile when the stent graft component is in the compressed configuration. In this embodiment, the elliptical opening stent 600 includes a body portion 602 and a spring portion 604. The body portion 602 has an even profile that allows the body stents to be crimped or wired to the body portion 602. The spring portion 604 urges the substantially elliptical opening to an open configuration. The elliptical opening stent 600 can be attached to the tubular graft by suture, adhesive, lamination, or the like. The elliptical opening stent 600 need not be affixed directly to the graft material of the tubular graft.

FIGS. 7-9 are additional embodiments of a stent graft component. FIG. 7 is a side view of a stent graft component with a graft flap. The graft flap 700 is a flap of graft material integrally joined to the tubular graft 702. The graft flap 700 extends outward near the leg intersection point 704 and beyond and around the support stent and springs of substantially elliptical opening 706. The graft flap 700 can assist in sealing around the substantially elliptical opening between the two stent graft components of the deployed stent graft system. In one embodiment, the graft flap of one stent graft component is operably connected to the other stent graft component with fasteners, such as staples, sutures, adhesives, rivets, screws, or the like. In one embodiment, the graft flap extends partially around the substantially elliptical opening.

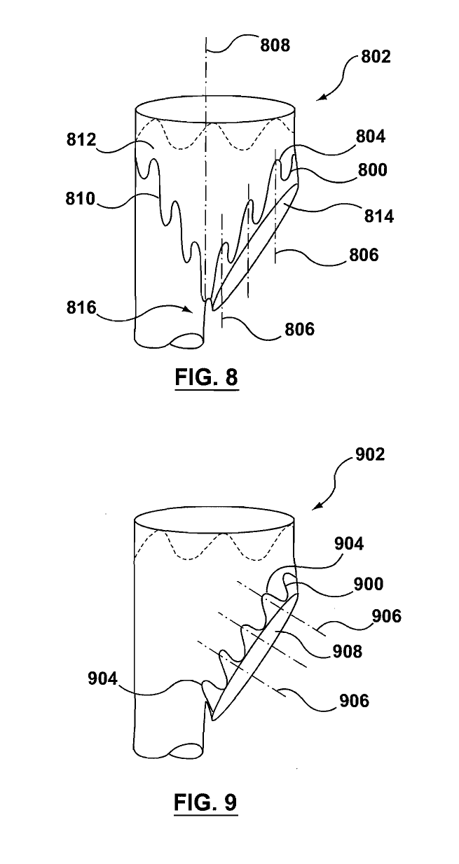

FIG. 8 is a side view of a stent graft component with an elliptical opening stent having longitudinally oriented peaks and a body elliptical stent. The elliptical opening stent 800 of the stent graft component 802 includes a number of peaks 804. The axes 806 of the peaks 804 are longitudinally oriented, which is defined as used herein as substantially oriented parallel to the longitudinal axis 808 of the stent graft component 802. In this embodiment, the stent graft component 802 also includes a body elliptical stent 810 operably connected around the body 812 opposite the substantially elliptical opening 814 and intersecting the leg intersection point 816. The body elliptical stent 810 in this example also has longitudinally oriented peaks and is symmetrical to the elliptical opening stent 800 across the axial mid-plane of the stent graft component 802. The body elliptical stent 810 in one stent graft component assists sealing about the other stent graft component in the deployed stent graft system.

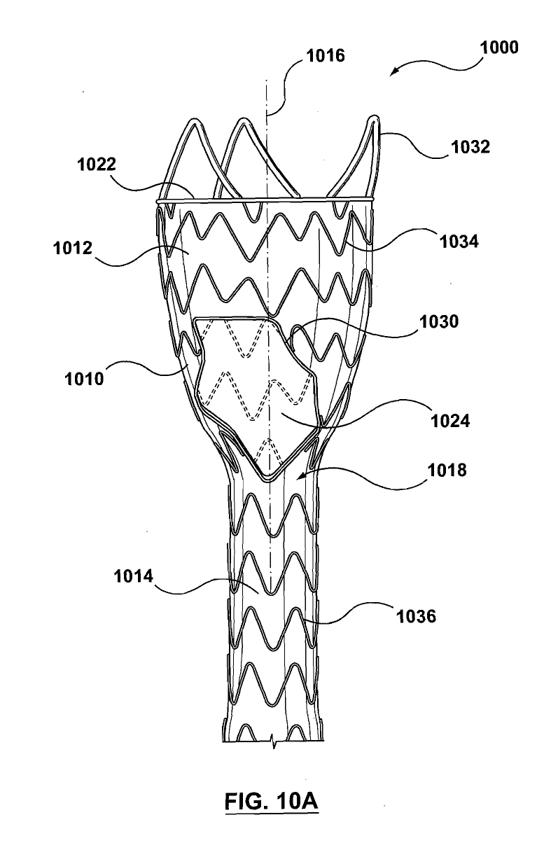

FIG. 9 is a side view of a stent graft component with an elliptical opening stent having peaks oriented orthogonally to the substantially elliptical opening. The elliptical opening stent 900 of the stent graft component 902 includes a number of peaks 904. The axes 906 of the peaks 904 are oriented orthogonally to the substantially elliptical opening 908.

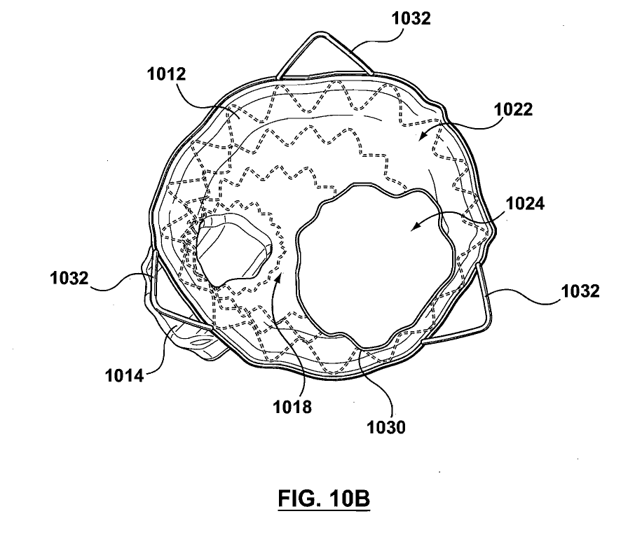

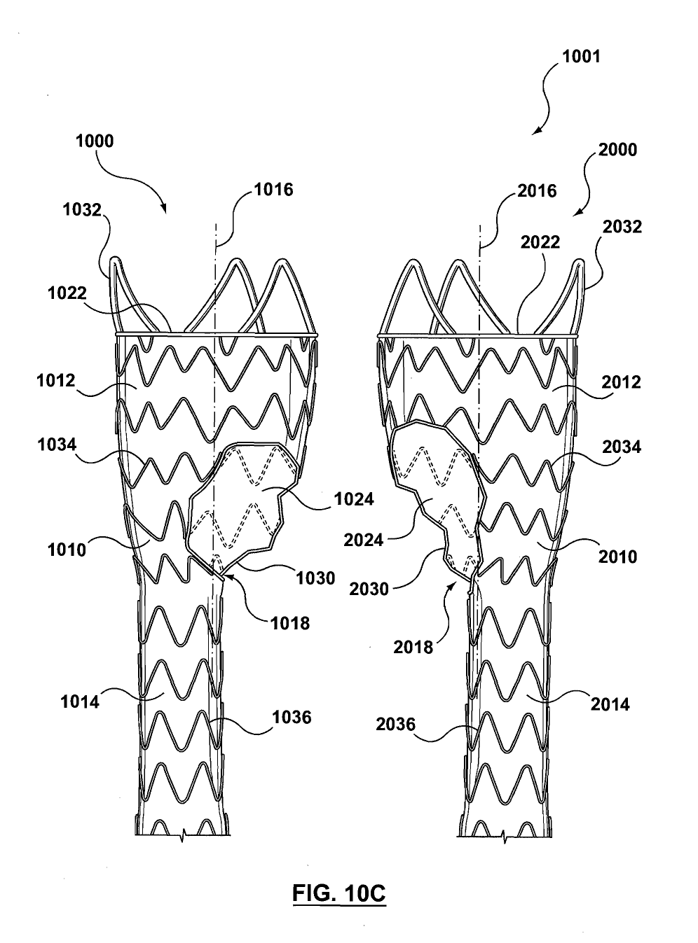

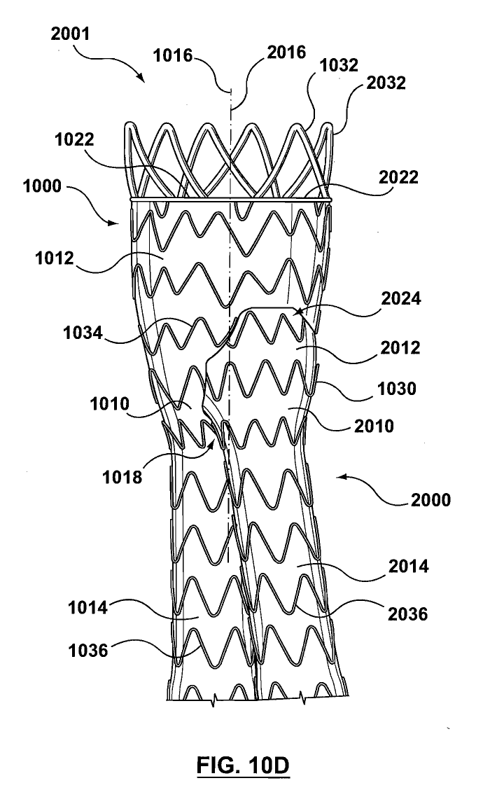

FIGS. 10A-10D, in which like elements share like reference numbers, are various views of an exemplary stent graft system. The figures illustrate an exemplary model of a stent graft system.

FIG. 10A is a side view of a stent graft component. The first stent graft component 1000 includes a tubular graft 1010, a crown stent 1032, body stents 1034, and leg stents 1036. The tubular graft 1010 has an integral body 1012 and leg 1014. The body 1012 has a longitudinal axis 1016 and a leg intersection point 1018 on or near the longitudinal axis 1016 where the leg 1014 meets the body 1012. The body 1012 defines a crown opening 1022 and a substantially elliptical opening 1024. The crown stent 1032 is operably connected around the crown opening 1022, the body stent 1034 is operably connected around the body 1012, and the leg stent 1036 is operably connected around the leg 1014. In this example, some of the body stents 1034 are attached to the perimeter 1030 of the substantially elliptical opening 1024, so the body stents acts as the elliptical opening stent.

FIG. 10B is a top view of a stent graft component. In this example, the crown stent 1032 includes an odd number of peaks around the perimeter 1021 of the crown opening 1022 and outside the body 1012. The peaks of one stent graft component are complementary with the peaks of the other stent graft component in the stent graft system, so the peaks follow the whole perimeter of the crown opening to form a continuous row of peaks when the stent graft system is assembled.

FIG. 10C is a side view of a stent graft system. The stent graft system 1001 includes first stent graft component 1000 and second stent graft component 2000. In this example, the first stent graft component 1000 is identical to the second stent graft component 2000. The second stent graft component 2000 includes a tubular graft 2010, an elliptical opening stent 2030, a crown stent 2032, a body stent 2034, and a leg stent 2036. The tubular graft 2010 has an integral body 2012 and leg 2014. The body 2012 has a longitudinal axis 2016 and a leg intersection point 2018 on the longitudinal axis 2016 where the leg 2014 meets the body 2012. The body 2012 defines a crown opening 2022 and a substantially elliptical opening 2024. The crown stent 2032 is operably connected around the crown opening 2022, the body stent 2034 is operably connected around the body 2012, and the leg stent 2036 is operably connected around the leg 2014. In this example, some of the body stents 2034 are attached to the perimeter 2030 of the substantially elliptical opening 2024, so the body stents acts as the elliptical opening stent.

FIG. 10D is a side view of an assembled stent graft system. The assembled stent graft system 2001 has body 2012 of the second stent graft component 2000 disposed in the body 1012 of the first stent graft component 1000 with the crown opening 2022 of the second stent graft component 2000 substantially aligned with the crown opening 1022 of the first stent graft component 1000. The second leg 2014 of the second stent graft component 2000 is disposed through the substantially elliptical opening 1024 of the first stent graft component 1000 with the perimeter of the first elliptical opening 1024 in contact with the body 2012 of the second stent graft component 2000. The longitudinal axis 1016 of the first stent graft component 1000 is substantially aligned with the longitudinal axis 2016 of the second stent graft component 2000. In this example, the peaks of the crown stent 1032 and the crown stent 2032 alternate around the perimeter of the crown opening.

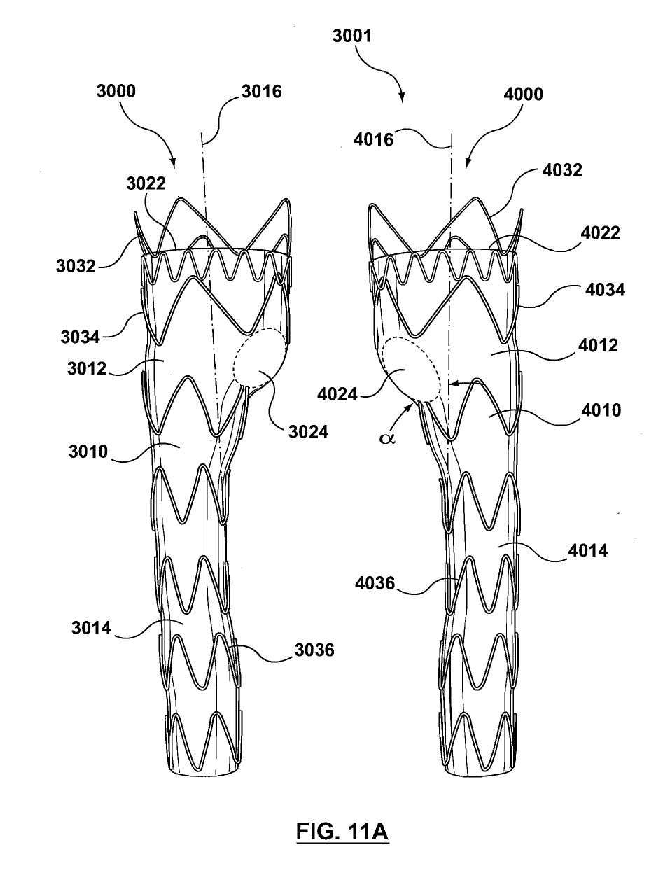

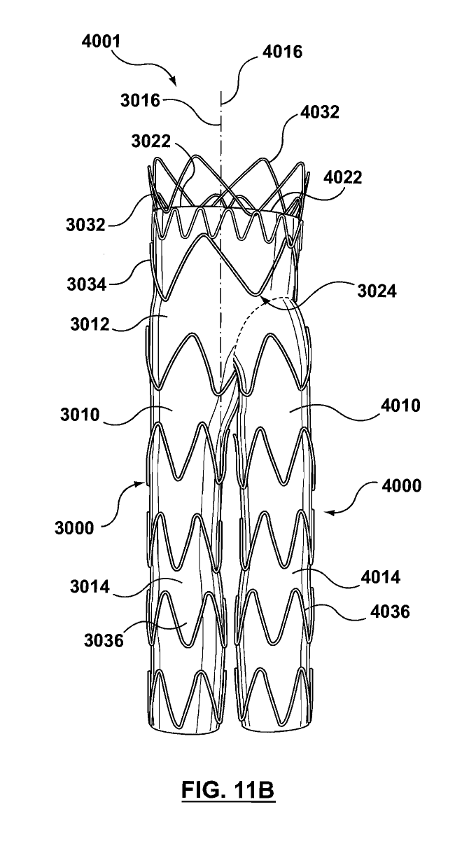

FIGS. 11A & 11B, in which like elements share like reference numbers, are side views of another exemplary stent graft system. In this example, the body of the stent graft component blends into the leg. The stent graft component in this example does not include a graft flap. Because of the blending, the longitudinal axis is defined as lying along the circumference of the leg and passing through the body. The acute angle between the substantially elliptical opening and the longitudinal axis is defined by the angle between the longitudinal axis and a line in the plane of and bisecting the substantially elliptical opening, although the substantially elliptical opening and the longitudinal axis may not intersect. In this example, no elliptical opening stent is placed around the substantially elliptical opening.

FIG. 11A is a side view of a stent graft system. The stent graft system 3001 includes first stent graft component 3000 and second stent graft component 4000. In this example, the first stent graft component 3000 is identical to the second stent graft component 4000.

The first stent graft component 3000 includes a tubular graft 3010, a crown stent 3032, body stents 3034, and leg stents 3036. The tubular graft 3010 has an integral body 3012 and leg 3014. The body 3012 has a longitudinal axis 3016 lying along the circumference of the leg 3014 and passing through the body 3012. The body 3012 defines a crown opening 3022 and a substantially elliptical opening 3024, the perimeter of which is illustrated by dashed lines. The crown stent 3032 is operably connected around the crown opening 3022, the body stent 3034 is operably connected around the body 3012, and the leg stent 3036 is operably connected around the leg 3014.

The second stent graft component 4000 includes a tubular graft 4010, an elliptical opening stent 4030, a crown stent 4032, a body stent 4034, and a leg stent 4036. The tubular graft 4010 has an integral body 4012 and leg 4014. The body 4012 has a longitudinal axis 4016 lying along the circumference of the leg 4014 and passing through the body 4012. The body 4012 defines a crown opening 4022 and a substantially elliptical opening 4024, the perimeter of which is illustrated by dashed lines. The longitudinal axis 4016 and the substantially elliptical opening 4024 are divided by an acute angle a. The crown stent 4032 is operably connected around the crown opening 4022, the body stent 4034 is operably connected around the body 4012, and the leg stent 4036 is operably connected around the leg 4014.

FIG. 11B is a side view of an assembled stent graft system. The assembled stent graft system 4001 has the body of the second stent graft component 4000 disposed in the body 3012 of the first stent graft component 3000 with the crown opening 4022 of the second stent graft component 4000 substantially aligned with the crown opening 3022 of the first stent graft component 3000. The second leg 4014 of the second stent graft component 4000 is disposed through the substantially elliptical opening 3024 of the first stent graft component 3000 with the perimeter of the first elliptical opening 3024 in contact with the body of the second stent graft component 4000. The longitudinal axis 3016 of the first stent graft component 3000 is substantially aligned with the longitudinal axis 4016 of the second stent graft component 4000. In this example, the peaks of the crown stent 3032 and the crown stent 4032 alternate around the perimeter of the crown opening.

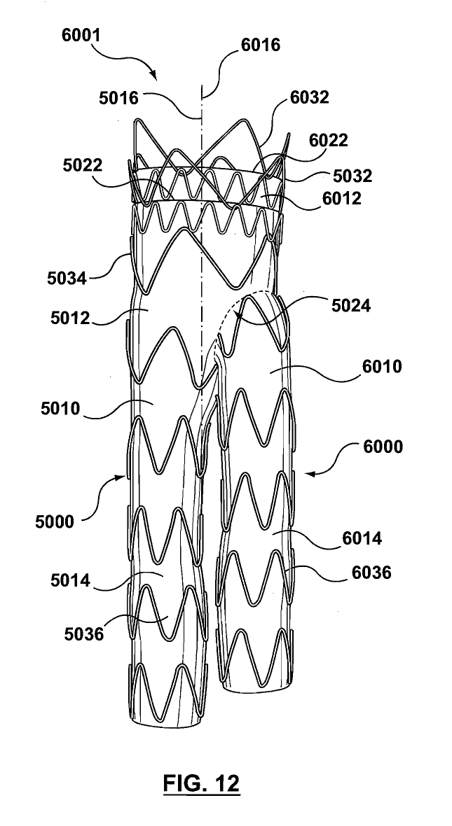

FIG. 12 is a side view of another exemplary assembled stent graft system. In this example, the body of one stent graft component is longer than the body of the other stent graft component, so the crown openings of the two stent graft components are not substantially aligned.

The first stent graft component 5000 includes a tubular graft 5010, a crown stent 5032, body stents 5034, and leg stents 5036. The tubular graft 5010 has an integral body 5012 and leg 5014. The body 5012 has a longitudinal axis 5016 lying along the circumference of the leg 5014 and passing through the body 5012. The body 5012 defines a crown opening 5022 and a substantially elliptical opening 5024, the perimeter of which is illustrated by dashed lines. The crown stent 5032 is operably connected around the crown opening 5022, the body stent 5034 is operably connected around the body 5012, and the leg stent 5036 is operably connected around the leg 5014.

The second stent graft component 6000 includes a tubular graft 6010, a crown stent 6032, body stents (not shown), and leg stents 6036. The tubular graft 6010 has an integral body 6012 and leg 6014. The body 6012 has a longitudinal axis 6016 lying along the circumference of the leg 6014 and passing through the body 6012. The body 6012 defines a crown opening 6022 and a substantially elliptical opening (not shown). The crown stent 6032 is operably connected around the crown opening 6022, the body stent (not shown) is operably connected around the body 6012, and the leg stent 6036 is operably connected around the leg 6014.

The assembled stent graft system 6001 has the body 6012 of the second stent graft component 6000 disposed in the body 5012 of the first stent graft component 5000 with the crown opening 6022 of the second stent graft component 6000 aligned longitudinally above the crown opening 5022 of the first stent graft component 5000. The second leg 6014 of the second stent graft component 6000 is disposed through the substantially elliptical opening 5024 of the first stent graft component 5000 with the perimeter of the first elliptical opening 5024 in contact with the body 6012 of the second stent graft component 6000. The longitudinal axis 5016 of the first stent graft component 5000 is substantially aligned with the longitudinal axis 6016 of the second stent graft component 6000.

While specific embodiments are disclosed herein, various changes and modifications can be made without departing from the spirit and scope of the invention.

1. A stent graft system comprising:
a first stent graft component comprising a first tubular graft having an integral first body and first leg, the first body having a first longitudinal axis, the first body defining a first crown opening and a first substantially elliptical opening, the first substantially elliptical opening being oriented at a first acute angle to the first longitudinal axis; and
a second stent graft component comprising a second tubular graft having an integral second body and second leg, the second body having a second longitudinal axis, the second body defining a second crown opening and a second substantially elliptical opening, the second substantially elliptical opening being oriented at a second acute angle to the second longitudinal axis;
wherein the second body is disposed in the first body, and the second leg is disposed through the first substantially elliptical opening with the perimeter of the first elliptical opening in contact with the second body.

2. The stent graft system of claim 1 wherein the first stent graft component further comprises a first elliptical opening stent operably connected to the first substantially elliptical opening, and the second stent graft component further comprises a second elliptical opening stent operably connected to the second substantially elliptical opening.

3. The stent graft system of claim 1 wherein the first stent graft component is identical to the second stent graft component.

4. The stent graft system of claim 1 wherein the second crown opening is substantially aligned with the first crown opening.

5. The stent graft system of claim 1 wherein friction between the second body and the first body holds the second body in place relative to the first body.

6. The stent graft system of claim 1 further comprising fasteners operably connecting the second body to the first body.

7. The stent graft system of claim 1 further comprising a graft flap integrally joined to the first tubular graft, the graft flap extending at least partially around the first substantially elliptical opening.

8. The stent graft system of claim 7 further comprising fasteners operably connecting the graft flap to the second stent graft component.

9. The stent graft system of claim 1 further comprising a body elliptical stent operably connected around the second body opposite the second substantially elliptical opening.

10. The stent graft system of claim 1 wherein the first stent graft component further comprises a first crown stent operably connected about the first crown opening and the second stent graft component further comprises a second crown stent operably connected about the second crown opening, the first crown stent includes first peaks outside the first body, the second crown stent includes second peaks outside the second body, and the first peaks are complementary with the second peaks to form a continuous row of peaks.

11. A stent graft comprising:
a tubular graft having an integral body and leg, the body having a longitudinal axis, the body defining a crown opening and a substantially elliptical opening, the substantially elliptical opening being oriented at an acute angle to the longitudinal axis;
an elliptical opening stent operably connected to the substantially elliptical opening; and
a crown stent operably connected about the crown opening.

12. The stent graft of claim 11 further comprising a graft flap integrally joined to the tubular graft, the graft flap extending at least partially around the substantially elliptical opening.

13. The stent graft of claim 11 further comprising a body stent operably connected around the body, wherein the elliptical opening stent is the body stent.

14. The stent graft of claim 11 wherein the elliptical opening stent is disposed around the substantially elliptical opening.

15. The stent graft of claim 14 further comprising a body stent operably connected around the body, wherein the elliptical opening stent is attached to the body stent.

16. The stent graft of claim 14 wherein the elliptical opening stent includes a spring portion.

17. The stent graft of claim 14 wherein the elliptical opening stent includes longitudinally oriented peaks.

18. The stent graft of claim 14 wherein the elliptical opening stent includes peaks oriented orthogonally to the substantially elliptical opening.

19. The stent graft of claim 12 further comprising a body elliptical stent operably connected around the body opposite the substantially elliptical opening.

20. The stent graft of claim 12 further comprising a body stent operably connected around the body and a leg stent operably connected around the leg.

21. The stent graft of claim 12 wherein the acute angle is between 20 degrees and 70 degrees.

22. A method of use of a stent graft system comprising:
positioning a first stent graft component in a first compressed configuration at a deployment site, the first stent graft component comprising:
a first tubular graft having an integral first body and first leg, the first body having a first longitudinal axis, the first body defining a first crown opening and a first substantially elliptical opening, the first substantially elliptical opening being oriented at a first acute angle to the first longitudinal axis; and
a first elliptical opening stent operably connected to the first substantially elliptical opening;
releasing the first stent graft component from the first compressed configuration to expand at the deployment site;
advancing a second stent graft component in a second compressed configuration through the first substantially elliptical opening to the deployment site, the second stent graft component comprising:
a second tubular graft having an integral second body and second leg, the second body having a second longitudinal axis, the second body defining a second crown opening and a second substantially elliptical opening, the second substantially elliptical opening being oriented at a second acute angle to the second longitudinal axis; and
a second elliptical opening stent operably connected to the second substantially elliptical opening;
releasing the second stent graft component from the second compressed configuration to expand with the second body within the first body and the second leg disposed through the first substantially elliptical opening with the perimeter of the first substantially elliptical opening in contact with the second body.

23. The method of claim 22 further comprising attaching the first stent graft component to the second stent graft component.

24. The method of claim 22 wherein the positioning comprises positioning the first stent graft component in a first compressed configuration at the deployment site through a first common iliac artery, and the advancing a second stent graft component comprises advancing the second stent graft component from a second common iliac artery through the first substantially elliptical opening.
